SEQUENCE LISTING (1 sequence)

SEQ ID NO: 1 - protein, 865 aa, Homo sapiens
MALVLGSLLLLGLCGNSFSGGQPSSTDAPKAWNYELPATNYETQDSHKAGPIGILFELVHIFLYVVQPRDFPEDTLRKFLQKAYESKIDYDKPETVILGLKIVYYEAGIILCCVLGLLFIILMPLVGYFFCMCRCCNKCGGEMHQRQKENGPFLRKCFAISLLVICIIISIGIFYGFVANHQVRTRIKRSRKLADSNFKDLRTLLNETPEQIKYILAQYNTTKDKAFTDLNSINSVLGGGILDRLRPNIIPVLDEIKSMATAIKETKEALENMNSTLKSLHQQSTQLSSSLTSVKTSLRSSLNDPLCLVHPSSETCNSIRLSLSQLNSNPELRQLPPVDAELDNVNNVLRTDLDGLVQQGYQSLNDIPDRVQRQTTTVVAGIKRVLNSIGSDIDNVTQRLPIQDILSAFSVYVNNTESYIHRNLPTLEEYDSYWWLGGLVICSLLTLIVIFYYLGLLCGVCGYDRHATPTTRGCVSNTGGVFLMVGVGLSFLFCWILMIIVVLTFVFGANVEKLICEPYTSKELFRVLDTPYLLNEDWEYYLSGKLFNKSKMKLTFEQVYSDCKKNRGTYGTLHLQNSFNISEHLNINEHTGSISSELESLKVNLNIFLLGAAGRKNLQDFAACGIDRMNYDSYLAQTGKSPAGVNLLSFAYDLEAKANSLPPGNLRNSLKRDAQTIKTIHQQRVLPIEQSLSTLYQSVKILQRTGNGLLERVTRILASLDFAQNFITNNTSSVIIEETKKYGRTIIGYFEHYLQWIEFSISEKVASCKPVATALDTAVDVFLCSYIIDPLNLFWFGIGKATVFLLPALIFAVKLAKYYRRMDSEDVYDDVETIPMKNMENGNNGYHKDHVYGIHNPVMTSPSQH

(19) United States
(12) Patent Application Publication
(10) Pub. No.: US 2008/0254488 A1
(43) Pub. Date: Oct. 16, 2008

(54) Cancer Test

(75) Inventor: Ruggero De Maria, Rome (IT)

Correspondence Address: GREENLEE WINNER AND SULLIVAN P C, 4875 PEARL EAST CIRCLE, SUITE 200, BOULDER, CO, 80301, US

(73) Assignee: Istituto Superiore di Sanita, Rome (IT)

(21) Appl. No.: 11/735,914

(22) Filed: Apr. 16, 2007

(51) Int. Cl. G01N 33/574; G01N 33/00; C12N 5/06

(52) U.S. Cl. 435/7.23; 435/7.92; 435/366

(57) ABSTRACT

The CD133 marker has been found to be diagnostic of malignant lung cancers. Tests and kits to show such cells and uses for such cells are disclosed.

### CROSS REFERENCE TO RELATED APPLICATIONS

Not applicable

### ACKNOWLEDGMENT OF FEDERAL RESEARCH FUNDING

Not applicable

### REFERENCE TO SEQUENCE LISTING

Not applicable

### BACKGROUND OF THE INVENTION

The present invention relates to testing for malignancy by assaying the presence of a cellular marker.

Lung cancer is the most common cause of cancer-related mortality worldwide. Four main categories of lung tumors contribute to the vast majority of cases in terms of both incidence and lethality. Small cell lung cancer (SCLC) is a neuroendocrine tumor that represents about 20 percent of all lung cancers, while the most common forms of the so called non-SCLC (NSCLC) include the adenocarcinoma (AC), squamous cell carcinoma (SCC) and large cell carcinoma (LCC) (1, 2). Despite the continuous efforts to improve the therapeutic response, the overall 5-year survival rate for such tumors is lower than 15% (3).

### SUMMARY OF THE INVENTION

Surprisingly, it has now been established that the malignancy of lung cancers is driven by a very small percentage of the cells making up the cancer, and that these cells are characterised by the presence of the CD133 marker. Cells carrying the CD133 marker are referred to herein as CD133+ cells.

Thus in a first aspect, the present invention provides a test for malignancy in a respiratory tract tissue sample, comprising assaying the sample for the presence of the CD133 marker.

Here, we found that the tumorigenic cells in small cell and non-small cell lung cancer are a rare population of undifferentiated cells expressing CD133, an antigen present on the cell membrane of normal and cancer primitive cells of the hematopoietic, neural, endothelial and epithelial lineages.

Thus, lung cancer contains a rare population of CD133+ cancer stem-like cells, able to self-renew and generate an unlimited progeny of non-tumorigenic cells.

In some embodiments, the test is for samples taken from the upper respiratory tract, the respiratory airways or lungs, such as the nose and nasal passages, paranasal sinuses, and throat or pharynx, the voice box or larynx, trachea, bronchi, bronchioles, respiratory bronchioles, alveolar ducts, alveolar sacs, and alveoli. In some embodiments, the test is for oral or lung cancer. In some embodiments, the test is for Small cell lung cancer (SCLC) and non-small cell lung cancers (NSCLC), including the adenocarcinoma (AC), squamous cell carcinoma (SCC) and large cell carcinoma (LCC).

In some embodiments, the test is considered positive for the existence of a cancerous condition when the amount of CD133+ cells in the sample is in excess of 1 CD133+ cell in 10³ cells, 10⁴ cells or 10⁵ cells.

In some embodiments, the cells in the sample are so treated as to separate individual cells in the sample. In some embodiments, the cells in the sample are so treated as to separate individual cells in the sample, such that at least 10%, 25% or 50% of the cells of the treated sample are associated with no more than one other cell. In some embodiments, the amount of cells specified is associated with no other cell.

In some embodiments, the presence of the CD133 marker is established by one or more means selected from microscopy, detection of radionuclides, detection of chromophores, ligand binding, magnetic labelling, enzyme linked immunoassay, immunofluorescence, chromatography, FACS (fluorescence activated cellular selection) and flow cytometry.

In another aspect, there is provided a method for establishing the presence of cancerous tissue in a sample from the respiratory tract, comprising contacting the sample with an optionally labelled anti-CD133 antibody, and establishing the level of binding of the antibody to the sample.

In some embodiments, the antibody is labelled such as to fluoresce, or be prepared in the form of microbeads, or may be unlabelled but detectable on a chromatographic column. Also provided is a kit comprising said antibodies, together with instructions for the use thereof in the establishment of the existence of a cancerous condition, or otherwise.

In another aspect, there is provided a method for cultivating CD133+ respiratory tumour cells, and especially CD133+ lung carcinoma cells, comprising the use of permissive conditions.

In a further aspect, there is provided a process for the selection of potential therapeutic agents, comprising the use of the cells of claim 17 to screen for therapeutic agents effective against cancers, especially those of the respiratory tract, and especially those tumours of whom the CD133+ cells form a part.

In a still further aspect, there is provided a method for selecting treatment for cancer of the respiratory tract according to the results obtained from a test as defined in claim 1.

### BRIEF DESCRIPTION OF THE DRAWINGS

FIG. 1. A small population of CD133+ tumor cells is present in lung cancer. Panel A) Immunohistochemistry for CD133 shows positive scattered cells within patient-derived SCC specimens. Right panel is a higher magnification of the region indicated in the left panel and allows improved visualization of CD133+ cells. Panel B) Absence of CD133+ cells within healthy lung tissue surrounding the tumor. Clearly visible alveoli, bronchus and blood vessel reveal standard morphology of healthy lung tissue (left panel). Right panel is a higher magnification of the region indicated in the left panel. Cells were counter-stained with hematoxylin. Panel C) Flow cytometry analysis of CD133 in 2 cases of freshly dissociated lung cancer samples. Percentages of CD133+ cells are indicated in control antibody (control) and specific antibody (CD133) stained samples. Panel D) Flow cytometry analysis of freshly dissociated lung cancer cells double stained with CD133PE and Ep-CAM-4FITC.

FIG. 2. Lung cancer tumorigenic cells are included in the CD133+ population. Panel A) Evaluation of the tumorigenic potential of freshly-isolated CD133⁺, CD133⁻ and unseparated (total) lung cancer cells after subcutaneous injection in matrigel. Data are mean ±s.d. of 3 independent experiments. Panel B) Hematoxylin-eosin analysis of colon cancer sections from the original tumors (patient) and corresponding xenografts (xenograft) obtained after injection of CD133+ from small cell lung cancer (SCLC) and non-SCLC (NSCLC). Original magnification was 20×.

FIG. 3. Establishment of lung cancer spheres of CD133+ cells. Panel A) Flow cytometry detection of CD133 in freshly dissociated lung tumor cells (fresh) or in the same cells cultured for the indicated times. Upper panels represent control antibody analysis of the corresponding CD133 stained cells in bottom panels. Panel B) Flow cytometric detection of carcinoembryonic (CEA), hematopoietic (CD45) or endothelial (CD31) antigens in lung cancer sphere-forming cells. All subtypes of lung cancer spheres displayed a similar expression for these antigens. Panel C) Phase contrast photographs of lung cancer spheres obtained from the indicated tumor subtypes (upper panels) and flow cytometry detection of the indicated antigens in the corresponding cells (lower panels). Grey histograms correspond to specific antibodies staining, white histograms represent negative control antibodies.

FIG. 4. In vitro differentiation potential of lung cancer spheres. Panel A) Microscopical analysis of lung cancer spheres grown as undifferentiated cells (spheres) or under differentiative conditions for two weeks (differentiated). (Lower panel) CD133 expression in the corresponding spheres and sphere-derived adherent progeny. Panels B and C) Expression of lung cell antigens in sphere-derived differentiated progeny analyzed by immunofluorescence (CKs and HMW-CKs) or flow cytometry (N-CAM).

FIG. 5. Lung cancer spheres are tumorigenic and reproduce the human tumor in immunocompromised mice. Hematoxylin-eosin (H&E) or immuno-histological staining for the indicated antigens performed on tumor specimens derived from parental tumor (patient) or from tumors generated by sub-cutaneous injection of lung cancer spheres in SCID mice (xenograft). The original magnification for each histological comparison is shown. Data are representative of four independent experiments.

FIG. 6. Self-renew and tumorigenic potential of CD133+ lung cancer cells before and after differentiation. Panel A) Extended proliferative capacity of lung cancer-derived spheres in comparison with their differentiated progeny. Growth curve values were obtained by cell counting at the indicated time points. SCC and AC-derived cells were used for the growth curve represented. Panel B) Tumorigenic potential of 10⁴ undifferentiated cells (spheres) as compared with 5×10⁴ cells after three weeks of differentiation (differentiated). Cells were simultaneously injected into the right and left flank of the same mouse, and mouse picture was taken 3 months after injection. Data shown in A and B are representative of four independent experiments. Panel C) Number of self-renewing cells in lung cancer spheres. Data represent the percentage of long-term growing cells in lung cancer spheres plated as single cell per well. Self-renewing cells were defined based on the percentage of clones showing exponential growth as secondary tumor spheres for more than five months. Data are the mean ±SD of three independent experiments.

### DETAILED DESCRIPTION OF THE INVENTION

The respiratory tract is divided into 3 segments, the upper respiratory tract, comprising the nose and nasal passages, paranasal sinuses, and throat or pharynx; the respiratory airways, comprising the voice box or larynx, trachea, bronchi, and bronchioles; and the lungs, comprising the respiratory bronchioles, alveolar ducts, alveolar sacs, and alveoli.

Thus, it will be appreciated that tissue samples may be taken from any part of the respiratory tract, so that the test may be to ascertain the existence of a cancerous condition in the tissues listed above, especially the mouth, throat, bronchioles, alveolar ducts, alveolar sacs, and alveoli, for example, and cancers tested for may include cancers associated with the above tissues, including oral, bronchiolar and lung cancers, for example. More specifically, it is particularly preferred that this test be used to determine the likelihood of existence of oral and lung cancer.

Polyps can occur in the respiratory tract, and particularly in the lung. However, many such polyps are benign, and may simply be removed by surgical means. What is important is to ascertain the existence of malignant cells in such polyps or other such respiratory tissue as may be subject to the test of the invention.

For convenience, tissues and samples will generally be referred to herein as being from the lung, but it will be appreciated that reference to the lung includes reference to any other part of the respiratory tract, including the mouth, unless otherwise indicated, or otherwise apparent from the context.

CD133 is a cell surface marker; it is also known as AC133, prominin 1, PROM1 or MSTP061, as indicated on the National Center for Biotechnology Information (NCBI) on the worldwide web. The protein sequence is given in NCBI Accession No. NP_{—}06008. Antibodies specific for this protein, especially the human protein, are commercially available, for example, from Miltenyi Biotec GmbH, Bergisch Gladbach, Germany or Abcam, Cambridge, England. Either monoclonal or polyclonal antibodies can be used in the practice of the present invention. See also Table 2.

By malignant is meant that attribute of cells in cancerous tissue that leads to the uncontrolled multiplication of undifferentiated cells. In the context of the present invention, it has been established that cells obtained from lung cancer tissue that do not express the CD133 marker are not capable of sustained existence beyond about two weeks, even under optimal conditions. By contrast, CD133+ cells are capable of replication under minimal conditions, and can readily generate new cancers in test animals.

The amount of cells in any given lung cancer that express the CD133 marker appears to be in the region of 5-30%, but this may be as low as 1 or 2.5% and as high as 50%, generally, and may vary beyond that under some circumstances. The observed percentage is higher when noncancerous cells, for example, fibroblasts, hematopoietic cells and endothelial cells, are removed from the tumor tissue prior to testing.

In the tests of the invention, a positive reading for the presence of a cancerous condition may be set at a level of anything in excess of 1 in 10³, but may be refined lower to 1 in 10⁴, or even 1 in 10⁵, CD133+ cells in a sample.

While it is possible to test untreated samples, it is generally preferred to disrupt the samples so as to separate the individual cells in the sample. Although it is not critical to completely disrupt the sample, it is preferred that at least 10% of the cells of the treated sample are associated with no more than one other cell, and are more preferably not bound to any other cell. More preferably, this amount is 25%, and yet more preferably 50%. While it is desirable that amounts of in excess of 50% are not associated with other cells, it is also preferred that the individual cells be not further disrupted, especially where a cell counting technique is used.

Disruption of samples may be by any conventional technique, and may involve physical means, as well as biological and chemical means. Thus, a combination of grinding and enzymes may be sufficient.

It will also be appreciated that a test of the present invention may involve simply the detection of CD133, and this may comprise total disruption of the sample, with or without subsequent purification, and detection of a representative amount of CD133 in the sample. This will generally require knowledge of the size of the sample and how much CD133 is normally present in non-cancerous tissue.

The disruption may also be to a lesser level, such that the cellular contents are disrupted and removed, leaving intact membranes, or “ghosts”, which may be labelled with anti-CD133 antibodies, for example, and subsequently counted.

The amount of CD133+ cells that exist in normal lung tissue is vanishingly small, to the extent that the detection of any cells whatsoever expressing CD133 is no small task even for the skilled person. Thus, merely establishing the presence of CD133+ cells by suitably labelled anti-CD133 antibodies will generally be indicative of a cancerous condition. The detection of cells expressing the CD133 marker may be by any suitable means, such as observation under a microscope, chromatography, FACS (fluorescence activated cellular selection) and flow cytometry, for example.

This process may be simplified such as by complete disruption of part of the sample and assaying for the presence of CD133 and, if CD133 is found, then employing more sophisticated techniques, such as FACS or flow cytometry, to ascertain the levels of CD133 marker expression in the remaining part of the sample.

The present invention further provides a method for establishing the presence of cancerous tissue in a sample from the respiratory tract, comprising contacting the sample with an optionally labelled anti-CD133 antibody, and establishing the level of binding of the antibody to the sample.

The antibody may be labelled such as to fluoresce, or be prepared in the form of microbeads, or may be unlabelled but detectable on a chromatographic column, for example.

The present invention further provides a kit comprising said antibodies, together with instructions for the use thereof in the establishment of the existence of a cancerous condition, or otherwise.

We have also demonstrated that it is possible to obtain a virtually unlimited expansion of lung cancer tumorigenic cells. This may be used in in vitro and in vivo evaluation of drug efficacy. In this context, the use of xenografts carrying a neoplastic lesion that closely resembles the original tumour is probably more reliable than cell line-based xenografts, and is the preferred technique to be used in optimising individualised therapies.

Thus, the present invention further provides cultivating CD133+ respiratory tumour cells, and especially CD133+ lung carcinoma cells, in permissive conditions. The Invention further provides the use of such cultivated cells to screen for therapeutic agents effective against cancers, especially those of the respiratory tract, and especially those tumours of whom the CD133+ cells form a part.

It will also be appreciated that detection of the tumorigenic CD133+ cells before and after treatment will provide an indicator as to the nature of the treatment necessary, both with regard to intensity and duration, as well as selection of treatment. With this knowledge, the skilled physician will be able to modify or select treatment according to numbers of CD133+ cells present before and after treatment.

Thus, the present invention further provides selecting treatment for cancer of the respiratory tract according to the results obtained from a test of the invention.

The present invention will now be further illustrated with reference to the following, non-limiting Example.

### EXAMPLE

Lung cancer is the most common cause of cancer-related mortality worldwide. Four main categories of lung tumors contribute to the vast majority of cases in terms of both incidence and lethality. Small cell lung cancer (SCLC) is a neuroendocrine tumor that represents about 20 percent of all lung cancers, while the most common forms of the so called non-SCLC (NSCLC) include the adenocarcinoma (AC), squamous cell carcinoma (SCC) and large cell carcinoma (LCC) (1, 2). Despite the continuous efforts to improve the therapeutic response, the overall 5-year survival rate for such tumors is lower than 15% (3).

Cancer stem cells are the rare population of undifferentiated tumorigenic cells responsible for tumor initiation, maintenance and spreading (4). These cells display unlimited proliferation potential, ability to self-renew and capacity to generate a progeny of differentiated cells that constitute the major tumor population. In light of the cancer stem cell-based model, normal stem cells might be considered as a proto-tumorigenic cells endowed with some properties typical of malignant cells, including the constitutive activation of survival pathways and the ability to proliferate indefinitely. Oncogenic mutations occurring in such a favorable background may turn the finely regulated growth potential of normal stem cells into the aberrant uncontrolled growth of cancer cells.

Cancer stem-like cells have been isolated and expanded from leukemia (5) and several solid tumors, including melanoma, breast, brain, prostate, pancreatic (6-13), and colon carcinomas (14, 15). These cells can be expanded in vitro as tumor spheres, while reproducing the original tumor when transplanted in immunodeficient mice.

The existence of human lung cancer stem cells has not been reported yet. However, indirect evidence suggests the possible presence of cancer stem cells in pulmonary tumors. Stem-like cells have been identified in mouse lung, such as a cell population able to drive the malignant transformation in experimentally-induced neoplasia (16). Moreover, human lung tumors sometimes show phenotypic heterogeneity, suggesting that they may originate from a multipotent cell (17).

Normal lung tissue is composed by a variety of cell types, such as basal mucous secretory cells of the trachea and bronchi, Clara cells of bronchioles, type 1 and type 2 pneumocytes of alveoli. These mature cells derive from the differentiation of lineage-restricted lung progenitor cells, which in turn originate from undifferentiated multipotent lung stem cells (18). Multipotent, long-lived cells have been identified throughout the airways and give rise to both transiently amplifying and terminally differentiated daughter cells. Like stem cells of other tissues, lung stem cells are responsible for local tissue maintenance and injury repair (19).

Some reports have described lung stem cells as cells expressing antigens typical of undifferentiated cells, such as CD34 and BCRP1 (20, 21). However, whether lung cancer might derive from the transformation of undifferentiated or differentiated cells remain to be elucidated.

### Materials and Methods

### Magnetic and Cytofluorimetric Cell Separation

For magnetic separation, cells were labeled 24-48 h after enzymatic dissociation with CD133/1 microbeads using the Miltenyi Biotec CD133 cell isolation kit. Alternatively, cells were labelled with CD133/1-PE antibody (Miltenyi Biotec) and sorted with a FACS Aria (Becton Dickinson). After magnetic or cytofluorimetric sorting, cell purity was evaluated by flow cytometry using CD133/2 (293C3)-PE or CD133/2 (293C3)-APC antibodies (Miltenyi Biotec).

### Isolation and Culture of Lung Cancer Spheres

Tumor samples were obtained in accordance with consent procedures approved by the Internal Review Board of Department of Laboratory Medicine and Pathology, Sant'Andrea Hospital, University La Sapienza, Rome. Surgical specimens were washed several times and left over night in DMEM:F-12 medium supplemented with high doses of Penicillin/Streptomycin and Amphotericin B in order to avoid contamination. Tissue dissociation was carried out by enzymatic digestion (20 μg/ml collagenase II, Gibco-Invitrogen, Carlsbad, Calif.) for 2 hours at 37° C. Recovered cells were cultured in serum-free medium containing 50 μg/ml insulin, 100 μg/ml apo-transferrin, 10 μg/ml putrescine, 0.03 mM sodium selenite, 2 μM progesterone, 0.6% glucose, 5 mM hepes, 0.1% sodium bicarbonate, 0.4% BSA, glutamine and antibiotics, dissolved in DMEM-F12 medium (Gibco-Invitrogen, Carlsbad, Calif.) and supplemented with 20 μg/ml EGF and 10 μg/ml bFGF. Flasks non-treated for tissue culture were used in order to reduce cell adherence and support growth as undifferentiated tumor-spheres. Medium was replaced or supplemented with fresh growth factors twice a week until cells started to grow forming floating aggregates. Cultures were expanded by mechanical dissociation of spheres, followed by re-plating of both single cells and residual small aggregates in complete fresh medium.

### Differentiation of Stem Cell Progeny

In order to obtain differentiation of lung cancer sphere-forming cells, stem cell medium was replaced with Bronchial Epithelial Cell Growth Medium (Cambrex, East Rutherford, N.J., USA) in tissue culture-treated flasks, to allow cell attachment and differentiation. The acquisition of differentiation markers and loss of stem cell markers was evaluated by flow cytometry or immunofluorescence as indicated above.

### Flow Cytometry and Immunofluorescence

For flow cytometry, tumor tissues or tumor-spheres were dissociated as single cells, washed and incubated with the appropriate dilution of control or specific antibody. Antibodies used were: PE-conjugated anti-CD133 from Miltenyi, anti-CD56/N-CAM (Neomarkers, Fremont, Calif.), FITC-conjugated anti Epithelial Membrane Antigen (Ep-CAM) (clone BerEP4), anti-human cytokeratins, anti-Carcinoembryonic Antigen, FITC-conjugated anti-CD34 and anti-CD45 (all from DAKO, Glostrup, Denmark), anti-CD31 (Beckton-Dickinson, Erembodegem, Belgium). After 45 minutes incubation cells were washed or, where necessary, incubated with FITC- or PE-conjugated secondary antibodies for 30 minutes and washed again before analysis using either a FACScan or an LSRII flow cytometer (Becton Dickinson). For immunofluorescence studies cells were grown on poly-D-lysine-coated glass coverslips, fixed with 2% paraformaldehyde for 20 min at 37° C. and permeabilized with 0.1% Triton X-100/PBS for 3 min at room temperature before incubation with the specific or control antibody. Stained cells were visualized with an Olympus confocal microscope.

### Immunohistochemistry on Tumor Sections

Immunohistochemistry was performed on formalin-fixed paraffin-embedded or frozen tissue. Five-micron paraffin sections were dewaxed in xylene and rehydrated with distilled water. Sections were treated with heat-induced epitope retrieval technique using a citrate buffer (pH6). For CD133 detection, epitope retrieval technique was based on EDTA (pH8). After peroxidase inhibition with 3% H₂O₂ for 20 minutes, the slides were incubated with the following antibodies: CD133/1 (Miltenyi Biotec, Bergisch Gladbach, Germany), low and medium molecular weight cytokeratins, High Molecular Weight Cytokeratins and Chromogranin A (DakoCytomation, Glostrup, Denmark) or N-CAM (Neomarkers, Fremont, Calif.). The reaction was performed using Elite Vector Stain ABC systems (Vector Laboratories) and DAB substrate chromogen (DakoCytomation), followed by counterstaining with haematoxylin.

### Cell Proliferation Assays

Spheres were plated at 10,000 cells/ml in growth medium supplemented with growth factors and after extended mechanical dissociation of culture aliquots, single cells were counted by trypan blue exclusion once a week. Adherent differentiated cells were plated in six well plates (10,000 cells/well) and one well every week was used for cell count. To determine their self-renewal ability, lung cancer cells were seeded in 96-well plates containing a single cell per well. Shortly after seeding, single cell-containing wells were identified and analyzed for the ability to generate long-term growing secondary spheres whose expansion was stable for more than 5 months.

Generation of Subcutaneous Lung Cancer Xenografts into SCID Mice

For mice xenografts, freshly isolated lung cancer cells or sphere-derived lung cancer cells were mixed with matrigel and injected subcutaneously. Four week-old female SCID or nude mice were used with similar results. Serial dilutions of cells (down to as low as 5×10³ cells) were injected in order to evaluate the tumorigenic activity of lung cancer CD133+ cells. Mice were monitored to check for the appearance of signs of disease, such as subcutaneous tumors or weight loss due to potential tumor growth in internal sites. When tumor diameters reached at least 5 mm in size, mice were sacrificed and tumor tissue was collected, fixed in buffered formalin and subsequently analyzed by immunohistochemistry. Hematoxylin and eosin staining followed by immunohistochemical analysis were performed to analyze tumor histology and to compare mouse xenografts with patient tumors.

### Results

CD133 Expressing Tumor Cells are Present within Lung Tumors with Variable Frequency

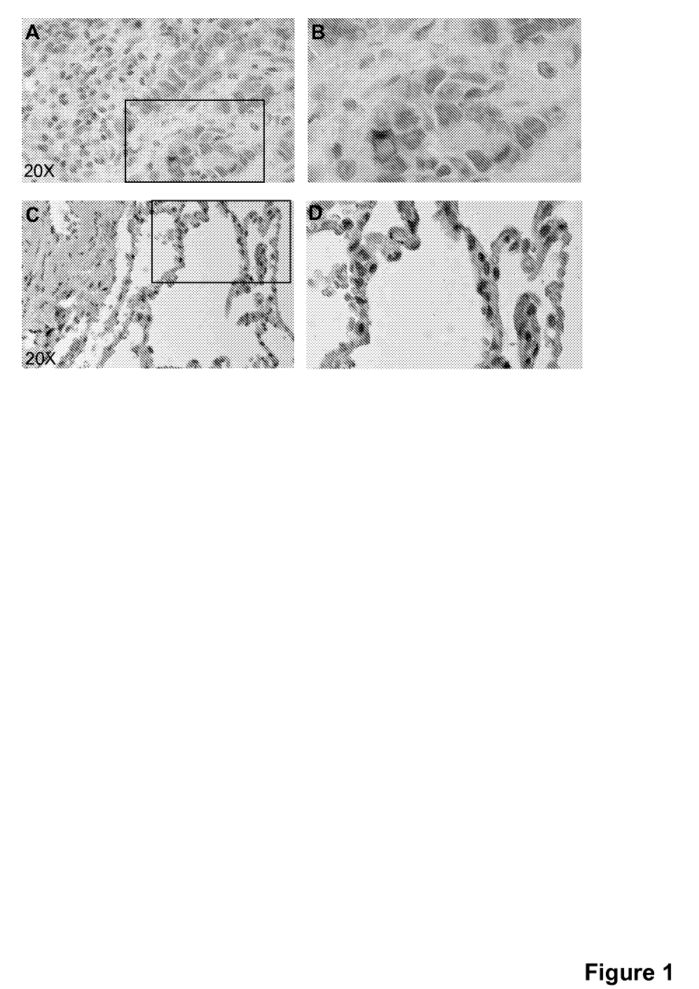

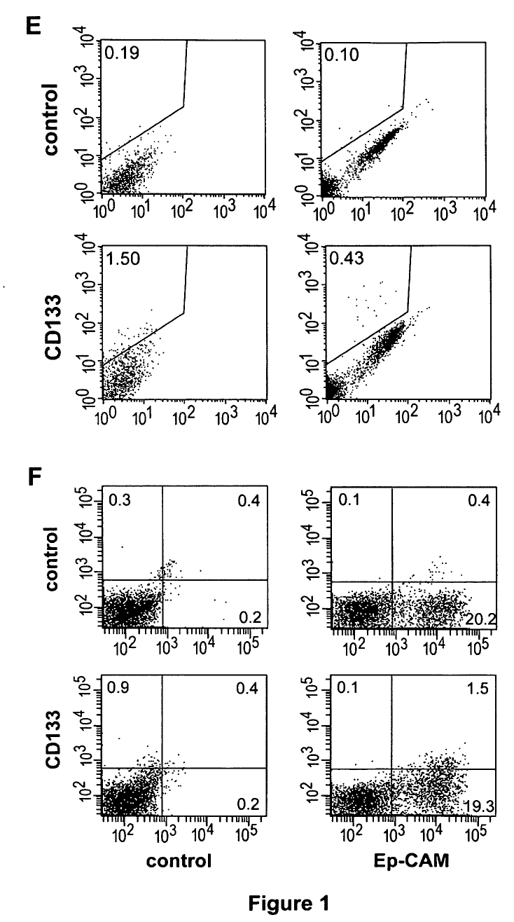

Several solid tumors contain CD133+ tumorigenic cancer stem cells, including glioblastoma, medulloblastoma and prostate carcinomas (7, 8, 11). Therefore, we first evaluated whether CD133+ cancer cells could be found within lung tumors. Immunohistochemical analysis performed on patient-derived tumor sections indicated the existence of rare CD133+ cells in lung tumors. The number of positive cells was low but variable among the different patients. In contrast, such CD133 expressing cells were barely detectable within healthy lung tissue specimens (FIG. 1A-B). In order to quantify more precisely the percentage of CD133+ cells, we analyzed enzymatically dissociated cancer tissues derived from both NSCLC and SCLC by flow cytometry. The three major types of NSCLC were examined, including SCC, AC and LCC. The latter displays a particular heterogeneity. Therefore, the analysis was restricted to one of the most frequent histological variant, the large cell neuroendocrine carcinoma (LCNEC). These experiments confirmed that the percentage of CD133+ cells was extremely low, with a few exceptions (FIG. 1C and Table 1).

TABLE 1
Case description and lung tumor sphere formation

| | Patient | | | CD133 | |
|---|---|---|---|---|---|
| Sample | (Sex/ | Tumor | TNM stage/ | expression | Sphere |
| N^{o} | Age) | subtype | grade | (%) | formation |
| 1 | M/73 | AC | pT2pN2pMX(IIIA) | 1.1 | no |
| | | | G3 | | |
| 2 | M/64 | AC | pT2pN1pMX(IIB) | <1 | No |
| | | | G2 | | |
| 3 | M78 | AC | pT2 pN2pMX/IIIA | <1 | Yes |
| | | | (G3) | | |
| 4 | F50 | AC | pT2pN0pMx/IB | <1 | No |
| | | | (G2) | | |
| 5 | F68 | AC | pT1 pN0 pMx/IA | <1 | No |
| | | | (G3) | | |
| 6 | M78 | AC | pT1pNXpMX | <1 | No |
| | | | G1 | | |
| 7 | M77 | AC | pT1pN0pMX/IA | <1 | No |
| | | | (G1) | | |
| 8 | F57 | AC | pT2pN0pMX/IB | <1 | No |
| | | | G1 | | |
| 9 | M70 | SCC | pT2 pN2 | 7 | Yes |
| | | | pMX/IIIA (G2) | | |
| 10 | M57 | SCC | ypT3pN0pMX/IIB | 1.7 | No |
| | | | (G2) | | |
| 11 | M65 | SCC | pT2pN0pMX/IB | <1 | No |
| | | | (G2/G3) | | |
| 12 | M73 | SCC | pT2pN0pM/IB | 1 | No |
| | | | G3 | | |
| 13 | M75 | SCC | pT2pN0pMX/IB | 1.1 | No |
| | | | G3 | | |
| 14 | M65 | SCLC | pT4pN2pMx/IIIB | 22 | Yes |
| 15 | M57 | SCLC | PT1pN2pMx/IA | <1 | Yes |
| 16 | F72 | mSCLC | pT3pN2pMX/IIIA | 2.9 | Yes |
| 17 | M57 | LCNEC | pT2pN0pMX/IB | <1 | No |
| | | | G3 | | |
| 18 | M63 | LCNEC | pT2pN2pMx/IIIA | 3.5 | Yes |
| | | | G3 | | |

Note:
Cells from freshly dissociated lung cancer tissues of four different sub-types (Adenocarcinoma, AC; Squamous Cell Carcinoma, SCC; Small Cell Lung Carcinoma, SCLC; and Large Cell NeuroEndocrine Carcinoma, LCNEC) were analyzed by flow cytometry for CD133 expression. The ability of the same samples to generate lung cancer spheres in vitro was evaluated by prolonged culture in growth factor-containing serum free medium.

No correlation was observed between the percentage of CD133+ cells and the tumor subtype. As for colon carcinoma, the vast majority of CD133+ cells in NSCLC expressed the epithelial antigen Ep-CAM, suggesting that the detection of such CD133+ population in lung carcinoma is due to the increased number of undifferentiated epithelial cells within the tumor (FIG. 1, Panel D). Thus, CD133+ cells were barely detectable in normal lung tissue, while representing a small but significant fraction of tumor cells.

### The Tumorigenic Population in Lung Cancer Express CD133

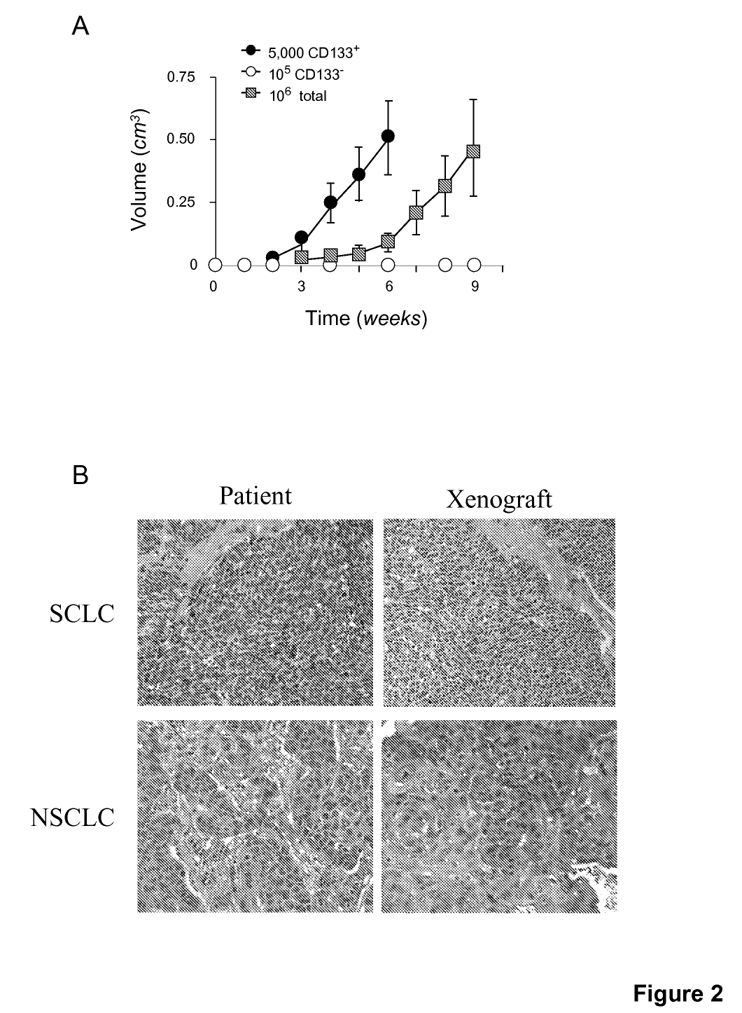

To identify the tumorigenic population responsible for lung cancer development and maintenance, CD133+ cells were isolated from NSCLC and SCLC, either by magnetic microbeads-linked anti-CD133 antibody or by flow cytometry sorting after labelling with PE-conjugated anti-CD133 antibody. While the injection of 10⁵ CD133− cells in SCID mice was unable to form subcutaneous tumor, the injection of 5×10³ CD133+ cells isolated from human lung tumors able to grow in such mice consistently generated tumor xenografts (FIG. 2A). Such tumors were histologically identical to the original human tumors from which the CD133+ cells were derived (FIG. 2, Panel B). Thus, the only tumorigenic cells in lung cancer are confined within the rare population expressing CD133, while all the other cancer cells not expressing CD133 are unable to form a tumor mass, suggesting that they have a limited growth potential and do not directly contribute to tumor growth and spreading.

Generation of CD133+ Lung Cancer Spheres from SCLC and NSCLC

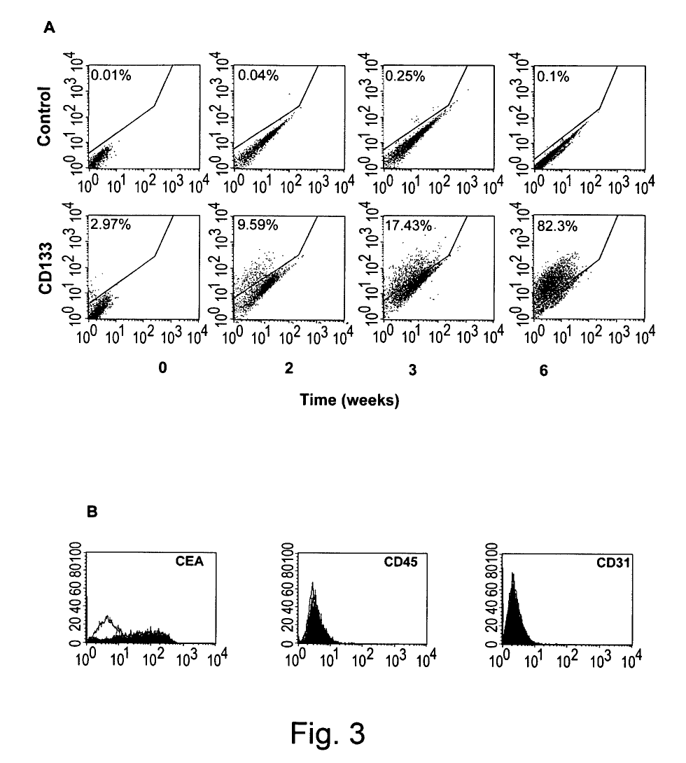

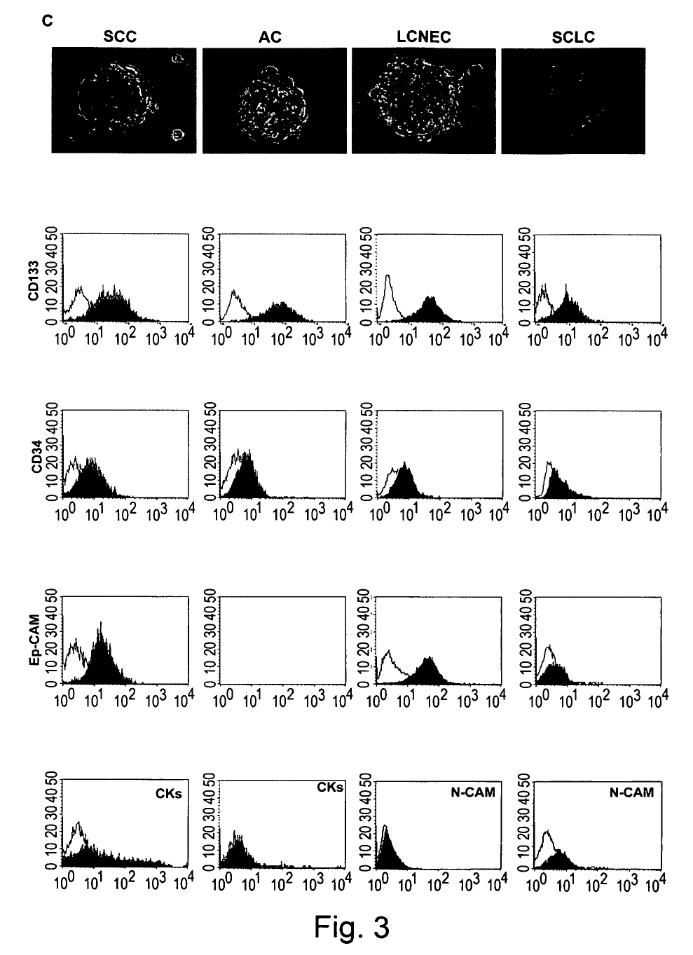

To determine whether lung cancer CD133+ cells can expand and generate long-term cultures in vitro, freshly-dissociated tumor cells from SCC, AC, LCNEC and SCLC were cultured at low density in serum-free medium containing EGF and basic FGF. As we previously showed, these culture conditions allowed the selection of undifferentiated colon or glioblastoma cancer stem and progenitor cells, while serum-dependent differentiated tumor cells and non-transformed accessory cells were negatively selected (14, 22). Exposure of lung cancer cells to such growth factors in the absence of serum allowed the selective growth of CD133+ cells, which increased in number (FIG. 3, Panel A) and gradually became a homogeneous population of undifferentiated cells expressing the carcinoembryonic antigen but not hematopoietic or endothelial markers (FIG. 3, Panel B). After approximately one or two months, these cell cultures became exclusively formed by cellular aggregates resembling the so-called “tumor-spheres” (FIG. 3, Panel C). About 30% of lung tumors gave rise to cancer spheres endowed with unlimited growth potential, being able to grow in culture for more than one year (Table 1 and data not shown). Cells from all the different subtypes of lung-cancer spheres consistently expressed high levels of CD133 and low levels of CD34. Non-SCLC (AC, SCC and LCNEC) spheres expressed considerable amounts of Ep-CAM, but not of cytokeratins (FIG. 3, Panel C), which are reportedly acquired during epithelial cell differentiation (23). Likewise, undifferentiated cells of both SCLC and the other tumor with neuroendocrine differentiation, LCNEC did not express significant levels of N-CAM, a key marker of neuroendocrine tumors (FIG. 3, Panel C). As for other tumor types, we obtained the formation of long-term growing spheres from a subset of tumors, suggesting that only 5 of the 15 lung tumors analyzed contain CD133+ cells able to grow in such culture conditions.

Lung Cancer Spheres Generate a Differentiated Progeny with Phenotypic Features of Lung Cancer Cells

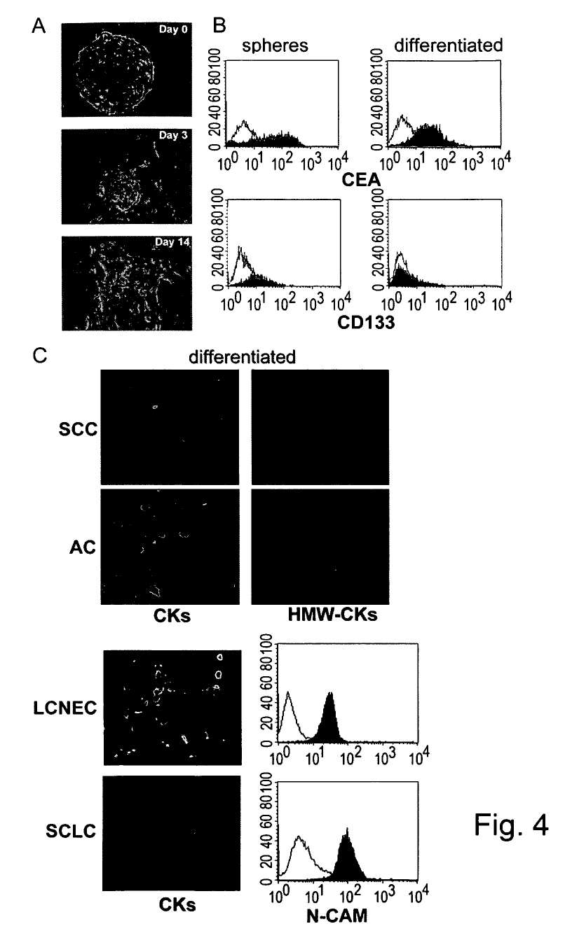

We next analyzed the in vitro differentiation potential of lung cancer spheres. In the presence of serum or specific medium for primary lung cell cultures, lung cancer spheres adhered to the plastic and acquired the typical morphologic features of differentiated cells (FIG. 4, Panel A). Both spheres and differentiated cells expressed CEA (results not shown), while the CD133 antigen was lost during differentiation, confirming the specific expression in undifferentiated cells (FIG. 4, Panel A). In contrast, the typical antigens found in the corresponding original tumors were gradually acquired after one week of culture. Specifically, we observed a considerable induction of N-CAM expression in the progeny of both large and small neuroendocrine lung cancer spheres (FIG. 4, Panel C). Low and medium molecular weight cytokeratins (CKs) were detected in all NSCLC (AC, SCC and LCNEC) cells, while the expression of high molecular weight cytokeratins (HMW-CKs) was induced only in SCC, indicating that lung cancer spheres are committed to produce a progeny of differentiated cells with phenotypic features of lung tumor cells (FIG. 4, Panels B and C). Thus, like cancer stem cells from other tumors (8, 9, 11, 14, 15), lung cancer spheres are composed by undifferentiated cells able to expand in the presence of EGF and basic FGF, but readily generating large and differentiated cells closely resembling the main cellular population of the original tumor under appropriate conditions.

### Lung Cancer Spheres are Tumorigenic In Vivo and Reproduce the Human Tumor

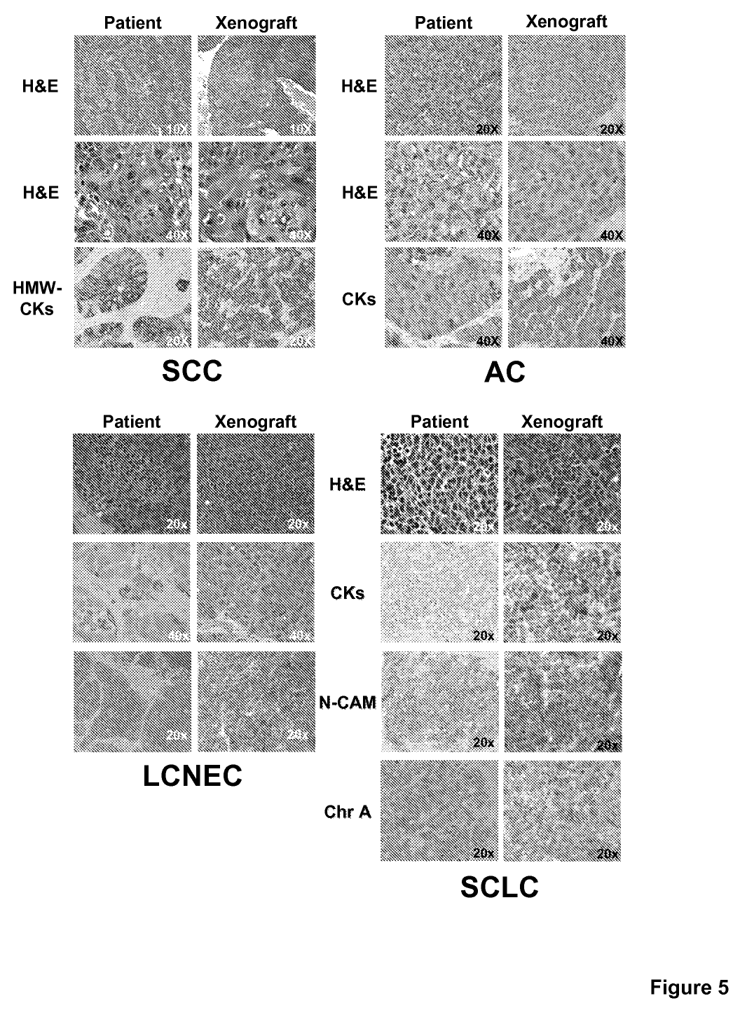

We next evaluated the tumorigenic potential of lung cancer CD133+ cells through subcutaneous injection in SCID mice of lung sphere cells mixed with growth factor-reduced matrigel. The injection of as low as 10⁴ CD133+ cells consistently resulted in growth of tumor xenografts with morphological features closely resembling the original tumor, as shown by hematoxylin and eosin staining (FIG. 5). In addition, immunohistochemistry of patient and mouse tumors showed that the immature malignant cells isolated from the different subtypes of lung cancers could generate mouse xenografts with antigen expression highly similar to the original tumor. Specifically, SCC displayed strong positivity for HMW-CKs (FIG. 5, Panel A), while all the NSCLC expressed CKs (FIG. 5, Panels A, B and C). Likewise, N-CAM was expressed by small and large cell neuroendocrine tumors, and SCLC xenografts expressed ChrA, another diagnostic marker for neuroendocrine tumors (FIG. 5, Panels C and D). Complete similarity between patient tumor and mouse xenograft was found for all antigens examined, demonstrating that tumor-spheres could effectively reproduce the human disease in the mouse. Importantly, mouse xenograft-derived cells could be serially transplanted in secondary and tertiary recipients, readily generating tumors with similar morphological and antigenic pattern (data not shown).

### Lung Cancer CD133+ Cells Loose the Self-Renew and Tumorigenic Potential Upon Differentiation

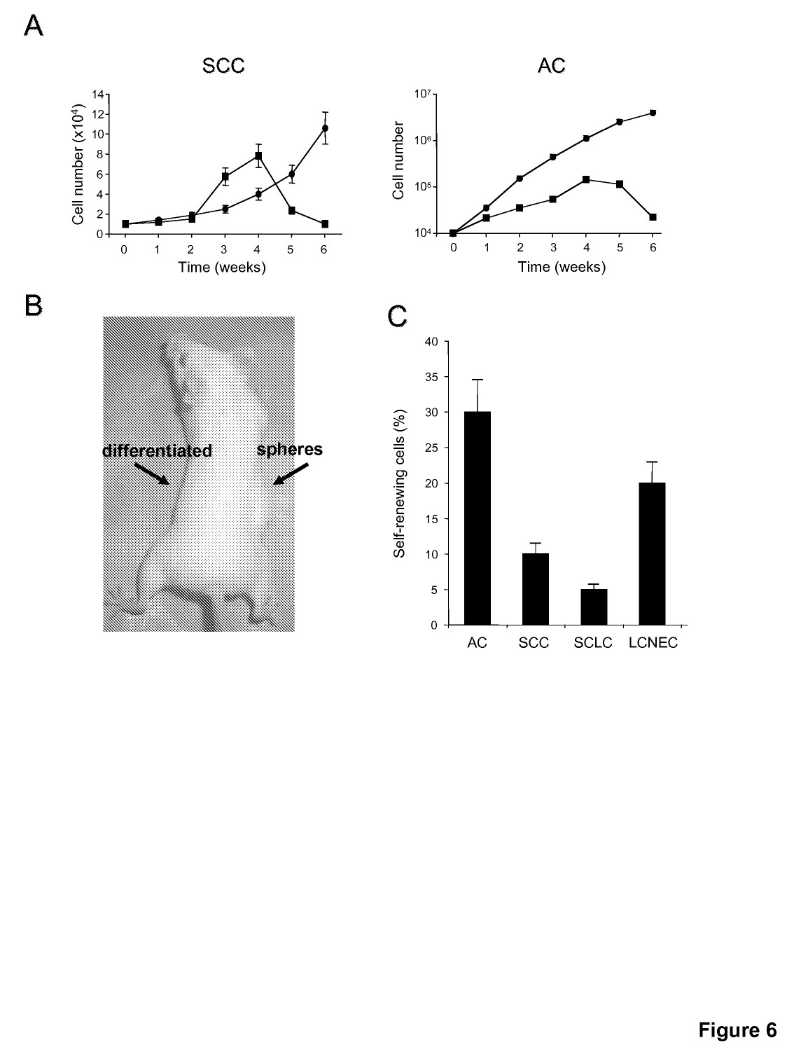

To confirm that lung cancer is initiated by a population of stem-like cells, we compared the growth potential of undifferentiated and differentiating cells. While lung cancer spheres displayed a stable exponential growth, differentiating tumor cells were able to proliferate for about four weeks before declining in number (FIG. 6, Panel A), suggesting that high proliferation potential of CD133+ lung cancer cells was lost during differentiation. To rule out the possibility that such a limited in vitro growth resulted from unfavourable culture conditions, we compared the tumorigenic potential of undifferentiated and differentiated cells in immunodeficient mice. While the subcutaneous injection of 10⁴ undifferentiated cells invariably produced tumor xenografts, a five fold higher number of actively proliferating differentiated cells was unable to generate tumors in SCID mice (FIG. 6, Panel B). Although cancer-derived spheres from single patients displayed a variable growth rate, all the samples analyzed underwent virtually unlimited expansion in vitro. The cell number required for tumor formation in mice did not increase after several passages in culture, indicating that the stem cell potential was not lost with extended proliferation (results not shown). To determine the percentage of putative cancer stem cells in lung spheres, we evaluated by limiting dilution analysis the ability of single cells to auto-replicate and generate new spheres endowed with unlimited growth potential in secondary cultures. We found that lung cancer spheres contained a high percentage of self-renewing cells, which ranged from 5 to 30% (FIG. 6, Panel C). Thus, lung cancer stem-like cells can be unlimitedly expanded and maintained in culture as tumor spheres containing a considerable percentage of tumorigenic cells.

### DISCUSSION

In spite of the variety of therapeutic attempts for the treatment of lung cancer, no major improvements in overall survival have been so far obtained. The identification and characterization of the tumorigenic population responsible for lung cancer formation and spreading may contribute to develop more effective therapies aiming at improving the prognosis of such severe condition.

Here, we identified the rare population of CD133+ cells as the lung cancer tumorigenic cell population. These cells were obtained from SCLC and several NSCLC subtypes through the use of selective culture conditions that allowed their expansion and characterization. Lung cancer spheres displayed undifferentiated cell phenotype revealed by CD133 expression and lack of lineage specific lung cell markers, suggesting that lung cancer could be initiated and propagated by undifferentiated stem-like cells.

Lung cancer CD133+ cells displayed the ability to generate differentiated lung cancer cells under appropriate culture conditions, as demonstrated by simultaneous acquisition of lineage specific markers and loss of CD133 expression. Such differentiated lung cancer cells were phenotypically very similar to the major cancer cell population present in the original tumor, indicating the existence of a precise hierarchical model for the generation of lung cancer tissue, based on the generation of a vast cell progeny by a small number of self-renewing undifferentiated cells.

Like tumorigenic cells from other tumors, lung cancer CD133+ cells were endowed with extensive proliferation and self-renewal potential, being able to grow as undifferentiated cells for more than 1 year without losing the ability to reproduce the original tumor after transplantation in immunocompromised animals. The number of self-renewing cells in lung cancer spheres ranges from 5 to 30%, as measured by clonogenic assays. Since CD133 expression is rather homogeneous in these cells, it is likely that lung cancer CD133+ cells comprise two populations of cells with similar phenotype, but different potential: a tumorigenic population of stem-like cells able to self-renew and a non-tumorigenic population of progenitor/precursor cells with a limited proliferation potential that constitute the early progeny of putative lung cancer stem cells.

Our in vivo studies confirmed the high tumorigenic potential of lung cancer spheres. A low number of lung cancer CD133+ cells was able to consistently generate tumor xenografts reproducing the original lung tumor both at morphologic and immunohistologic level. This system may provide an excellent model to study lung cancer biology and its response to therapeutic approaches at pre-clinical level.

Based on results obtained with cells surviving lung-damaging agents, two subtypes of Clara cells were identified in mice as the major lung reparative populations resident in neuroepithelial bodies and at the bronchoalveolar duct junction. Neuroepithelial bodies are scattered islands of amine and peptide containing vesicles that are released upon hypoxic stimulation. Surviving Clara cells of the neuroepithelial bodies can replenish both the neuroendocrine and epithelial cell population (24). The second subtype of surviving Clara cells is located at the bronchoalveolar duct junction and seems to play a key role in the regeneration of the epithelial components of the bronchoalveolar structure (25). These reparative bronchoalveolar cells are able to self-renew and display all the features of a regional stem cell of the distal lung (16). Expression of K-ras promoted the transformation of mouse bronchoalveolar stem cells, which gave rise to lung adenocarcinomas after naphthalene treatment, suggesting that bronchoalveolar stem cells could be a target of tumor transformation in lung cancer (16).

Although there is not a full overlap between human and mouse stem cell antigens, human lung cancer CD133+ cells and mouse bronchoalveolar stem cells share the expression of the stem cell marker CD34 and the absence of pan-hematopoietic (CD45) and -endothelial (CD31) antigens (16). The extreme rarity of CD133+ cells in normal lung tissue is compatible with the hypothesis that a minute number of stem cells reside in organs at slow cell turnover, such as lung (26).

Lung cancer spheres contain a significant percentage of stem-like cells able to self-renew. The availability of tumorigenic lung cancer cell culture may considerably contribute to the understanding of lung cell biology. Extensive phenotyping and characterization of CD133+ lung cancer cells may provide key information on relevant pathways to be targeted to increase the therapeutic response. Likewise, the possibility to detect the tumorigenic population may facilitate the development of new diagnostic and prognostic procedures. In this context, the use of preclinical models based on the use of primary lung tumorigenic cells may represent a considerable tool to obtain new advances to be exploited in the clinical setting.

All references cited in this application, for example, patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

All patents and publications mentioned in the specification reflect the levels of skill of those skilled in the art to which the invention pertains. References cited herein are incorporated by reference herein in their entirety to indicate the state of the art, in some cases as of their filing date, and it is intended that this information can be employed herein, if needed, to exclude (for example, to disclaim) specific embodiments that are in the prior art. For example, when a compound is claimed, it should be understood that compounds known in the prior art, including certain compounds disclosed in the references disclosed herein (particularly in referenced patent documents), are not intended to be included in the claim.

When a compound is claimed, it should be understood that compounds known in the art including the compounds disclosed in the references disclosed herein are not intended to be included. When a Markush group or other grouping is used herein, all individual members of the group and all combinations and subcombinations possible of the group are intended to be included in the disclosure as well.

Every formulation or combination of components described or exemplified can be used to practice the invention, unless otherwise stated. Specific names of compounds are intended to be exemplary, as it is known that one of ordinary skill in the art can name the same compounds differently. One of ordinary skill in the art will appreciate that methods, starting materials, antibodies, and means and compositions for detecting relevant and specific antibody binding other than those specifically exemplified can be employed in the practice of the invention without resort to undue experimentation. All art-known functional equivalents of any such methods, elements, starting materials, and tissue sample collection, cell dispersion and detection methods are intended to be encompassed by the present invention. Whenever a range is given in the specification, for example, a temperature range, a time range, or a composition range, all intermediate ranges and subranges, as well as all individual values included in the ranges given, are intended to be included in the disclosure.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. Any recitation herein of the term “comprising”, particularly in a description of components of a composition or in a description of elements of a device, is understood to encompass those compositions and methods consisting essentially of and consisting of the recited components or elements. The invention illustratively described herein suitably may be practiced in the absence of any element or elements, limitation or limitations which is not specifically disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

In general the terms and phrases used herein have their art-recognized meanings, which can be found by reference to standard texts, journal references and contexts known to those skilled in the art, for example, in the references cited herein.

One skilled in the art readily appreciates that the present invention is well adapted to carry out the objects and obtain the ends and advantages mentioned, as well as those inherent in the present invention. The methods, components, materials and dimensions described herein as currently representative of preferred embodiments are provided as examples and are not intended as limitations on the scope of the invention. Changes therein and other uses which are encompassed within the spirit of the invention will occur to those skilled in the art, are included within the scope of the claims.

Although the description herein contains certain specific information and examples, these should not be construed as limiting the scope of the invention, but as merely providing illustrations of some of the embodiments of the invention. Thus, additional embodiments are within the scope of the invention and within the following claims.

TABLE 2
Abcam Anti-CD133 antibodies

| Code | Name | Raised In | Clonality | Applications | Info |
|---|---|---|---|---|---|
| ab16518 | CD133 | Rabbit | Polyclonal | ELISA, | Synthetic peptide: |
| | antibody | | | ICC/IF, WB | KDHVYGIHNPVMTSPSQH, |
| | | | | | corresponding to C terminal |
| | | | | | amino acids 848-865 of CD133, |
| | | | | | SEQ ID NO: 1. |
| | Reacts with Human and Mouse. Not yet tested in other species. Predicted to react | | | | |
| | with Rat (87% identity with immunogen) due to sequence homology. | | | | |
| ab19898 | CD133 | Rabbit | Polyclonal | ICC/IF, WB | Synthetic peptide conjugated to |
| | antibody - | | | | KLH derived from within residues |
| | Stem Cell | | | | 800 to the C-terminus of Human |
| | Marker | | | | CD133, SEQ ID NO: 1. |
| | Reacts with Human and Mouse. Not yet tested in other species. Predicted to react | | | | |
| | with Rat (87% identity with immunogen) due to sequence homology. | | | | |
| ab5558 | CD133 | Mouse | Monoclonal | ELISA, Flow | Synthetic peptide: GGQPSSTDAPK |
| | antibody | | | Cyt, WB | AWNYEL |
| | [32AT1672] | | | | conjugated to KLH, |
| | | | | | corresponding to amino acids 20-36 |
| | | | | | of Human CD133, SEQ ID |
| | | | | | NO: 1. |
| | Reacts with Human. Not yet tested in other species. | | | | |
| ab31448 | CD133 | Rabbit | Polyclonal | ICC/IF, WB | Synthetic peptide conjugated to |
| | antibody - | | | | KLH derived from within residues |
| | Extracellular | | | | 350-450 of Mouse CD133, |
| | domain | | | | within an extracellular region. |

Reacts with Mouse. Not yet tested in other species. Based on the immunogen
sequence, this antibody is not expected to recognise CD133 in species other than
mouse.
ICC/IF = Immunocytochemistry/Immunofluorescence.
WB = Western blot.

Sequence of CD133 (human) from NP_006008
(SEQ ID NO: 1)
(National Center for Biotechnology Information,
website):

| | |
|---|---|
| 1 | malvlgslll lglcgnsfsg gqpsstdapk awnyelpatn |
| | yetqdshkag pigilfelvh |
| 61 | iflyvvqprd fpedtlrkfl qkayeskidy dkpetvilgl |
| | kivyyeagii lccvlgllfi |
| 121 | ilmplvgyff cmcrccnkcg gemhqrqken gpflrkcfai |
| | sllviciiis igifygfvan |
| 181 | hqvrtrikrs rkladsnfkd lrtllnetpe qikyilaqyn |
| | ttkdkaftdl nsinsvlggg |
| 241 | ildrlrpnii pvldeiksma taiketkeal enmnstlksl |
| | hqqstqlsss ltsvktslrs |
| 301 | slndplclvh pssetcnsir lslsqlnsnp elrqlppvda |
| | eldnvnnvlr tdldglvqqg |
| 361 | yqslndipdr vqrqtttvva gikrvlnsig sdidnvtqrl |
| | piqdilsafs vyvnntesyi |
| 421 | hrnlptleey dsywwlgglv icslltlivi fyylgllcgv |
| | cgydrhatpt trgcvsntgg |
| 481 | vflmvgvgls flfcwilmii vvltfvfgan vekilcepyt |
| | skelfrvldt pyllnedwey |
| 541 | ylsgklfnks kmkltfeqvy sdckknrgty gtlhlqnsfn |
| | isehlnineh tgsisseles |
| 601 | lkvnlnifll gaagrknlqd faacgidrmn ydsylaqtgk |
| | spagvnllsf aydleakans |
| 661 | lppgnlrnsl krdaqtikti hqqrvlpieq slstlyqsvk |
| | ilqrtgngll ervtrilasl |
| 721 | dfaqnfitnn tssviieetk kygrtiigyf ehylqwiefs |
| | isekvasckp vataldtavd |
| 781 | vflcsyiidp lnlfwfgigk atvfllpali favklakyyr |
| | rmdsedvydd vetipmknme |
| 841 | ngnngyhkdh vygihnpvmt spsqh |

### Rat and Mouse Homologues of Human CD133

Homologues from Rattus norvegicus and Mus musculus can be found at NCBI Accession Numbers ABI50090 and NP_{—}032961, respectively, where CD133 is known as prominin 1.

### REFERENCES

1. Collins, L. G., Haines, C., Perkel, R., and Enck, R. E. Lung cancer: diagnosis and management. Am Fam Physician, 75: 56-63, 2007.
2. Petersen, I. and Petersen, S. Towards a genetic-based classification of human lung cancer. Anal Cell Pathol, 22: 111-121, 2001.
3. Jemal, A., Siegel, R., Ward, E., Murray, T., Xu, J., Smigal, C., and Thun, M. J. Cancer statistics, 2006. CA Cancer J Clin, 56: 106-130, 2006.
4. Pardal, R., Clarke, M. F., and Morrison, S. J. Applying the principles of stem-cell biology to cancer. Nat Rev Cancer, 3: 895-902, 2003.
5. Bonnet, D. and Dick, J. E. Human acute myeloid leukemia is organized as a hierarchy that originates from a primitive hematopoietic cell. Nat Med, 3: 730-737, 1997.
6. Al-Hajj, M., Wicha, M. S., Benito-Hernandez, A., Morrison, S. J., and Clarke, M. F. Prospective identification of tumorigenic breast cancer cells. Proc Natl Acad Sci USA, 100: 3983-3988, 2003.
7. Patrawala, L., Calhoun, T., Schneider-Broussard, R., Li, H., Bhatia, B., Tang, S., Reilly, J. G., Chandra, D., Zhou, J., Claypool, K., Coghlan, L., and Tang, D. G. Highly purified CD44+ prostate cancer cells from xenograft human tumors are enriched in tumorigenic and metastatic progenitor cells. Oncogene, 25: 1696-1708, 2006.
8. Singh, S. K., Clarke, I. D., Terasaki, M., Bonn, V. E., Hawkins, C., Squire, J., and Dirks, P. B. Identification of a cancer stem cell in human brain tumors. Cancer Res, 63: 5821-5828, 2003.
9. Singh, S. K., Hawkins, C., Clarke, I. D., Squire, J. A., Bayani, J., Hide, T., Henkelman, R. M., Cusimano, M. D., and Dirks, P. B. Identification of human brain tumour initiating cells. Nature, 432: 396-401, 2004.
10. Fang, D., Nguyen, T. K., Leishear, K., Finko, R., Kulp, A. N., Hotz, S., Van Belle, P. A., Xu, X., Elder, D. E., and Herlyn, M. A tumorigenic subpopulation with stem cell properties in melanomas. Cancer Res, 65: 9328-9337, 2005.
11. Galli, R., Binda, E., Orfanelli, U., Cipelletti, B., Gritti, A., De Vitis, S., Fiocco, R., Foroni, C., Dimeco, F., and Vescovi, A. Isolation and characterization of tumorigenic, stem-like neural precursors from human glioblastoma. Cancer Res, 64: 7011-7021, 2004.
12. Prince, M. E., Sivanandan, R., Kaczorowski, A., Wolf, G. T., Kaplan, M. J., Dalerba, P., Weissman, I. L., Clarke, M. F., and Ailles, L. E. Identification of a subpopulation of cells with cancer stem cell properties in head and neck squamous cell carcinoma. Proc Natl Acad Sci USA, 104: 973-978, 2007.
13. Li, C., Heidt, D. G., Dalerba, P., Burant, C. F., Zhang, L., Adsay, V., Wicha, M., Clarke, M. F., and Simeone, D. M. Identification of pancreatic cancer stem cells. Cancer Res, 67:1030-1037, 2007.
14. Ricci-Vitiani, L., Lombardi, D. G., Pilozzi, E., Biffoni, M., Todaro, M., Peschle, C., and De Maria, R. Identification and expansion of human colon-cancer-initiating cells. Nature, 2006.
15. O'Brien C, A., Pollett, A., Gallinger, S., and Dick, J. E. A human colon cancer cell capable of initiating tumour growth in immunodeficient mice. Nature, 2006.
16. Kim, C. F., Jackson, E. L., Woolfenden, A. E., Lawrence, S., Babar, I., Vogel, S., Crowley, D., Bronson, R. T., and Jacks, T. Identification of bronchioalveolar stem cells in normal lung and lung cancer. Cell, 121: 823-835, 2005.
17. Berns, A. Stem cells for lung cancer? Cell, 121: 811-813, 2005.
18. Otto, W. R. Lung epithelial stem cells. J Pathol, 197: 527-535, 2002.
19. Giangreco, A., Groot, K. R., and Janes, S. M. Lung Cancer and Lung Stem Cells Strange Bedfellows? Am J Respir Crit Care Med, 2006.
20. Summer, R., Kotton, D. N., Sun, X., Ma, B., Fitzsimmons, K., and Fine, A. Side population cells and Bcrp1 expression in lung. Am J Physiol Lung Cell Mol Physiol, 285: L97-104, 2003.
21. Summer, R., Kotton, D. N., Sun, X., Fitzsimmons, K., and Fine, A. Translational physiology: origin and phenotype of lung side population cells. Am J Physiol Lung Cell Mol Physiol, 287: L477-483, 2004.
22. Eramo, A., Ricci-Vitiani, L., Zeuner, A., Pallini, R., Lotti, F., Sette, G., Pilozzi, E., Larocca, L. M., Peschle, C., and De Maria, R. Chemotherapy resistance of glioblastoma stem cells. Cell Death Differ, 13: 1238-1241, 2006.
23. Moll, R. Cytokeratins as markers of differentiation in the diagnosis of epithelial tumors. Subcell Biochem, 31: 205-262, 1998.
24. Reynolds, S. D., Giangreco, A., Power, J. H., and Stripp, B. R. Neuroepithelial bodies of pulmonary airways serve as a reservoir of progenitor cells capable of epithelial regeneration. Am J Pathol, 156: 269-278, 2000.
25. Giangreco, A., Reynolds, S. D., and Stripp, B. R. Terminal bronchioles harbor a unique airway stem cell population that localizes to the bronchoalveolar duct junction. Am J Pathol, 161: 173-182, 2002.
26. Rawlins, E. L. and Hogan, B. L. Epithelial stem cells of the lung: privileged few or opportunities for many? Development, 133: 2455-2465, 2006.

1. A test for malignancy in a respiratory tract tissue sample, comprising detecting CD133 marker protein in the sample.

2. The test of claim 1, wherein the assaying is by contacting the sample with an antibody specific for CD133 under conditions allowing binding of antibody specific for CD133 with a CD133 protein present in the sample.

3. The test of claim 1, wherein the sample is taken from the upper respiratory tract, the respiratory airways or lung.

4. The test of claim 1, wherein the cancer tested for is small cell lung cancer (SCLC) and a non-small cell lung cancer (NSCLC), including the adenocarcinoma (AC), squamous cell carcinoma (SCC) and large cell carcinoma (LCC),

5. The test of claim 1, wherein the cancer tested for is cancer of the nose and nasal passages, paranasal sinuses, throat or pharynx, the voice box or larynx, trachea, bronchi, bronchioles, respiratory bronchioles, alveolar ducts, alveolar sacs, and alveoli.

6. The test of claim 1, wherein the cancer tested for is oral or lung cancer.

7. The test of claim 1, wherein the test is considered positive for the existence of a cancerous condition when the amount of CD133+ cells in the sample is in excess of 1 CD133+ cell in 10³ total cells.

8. The test of claim 1, wherein the test is considered positive for the existence of a cancerous condition when the amount of CD133+ cells in the sample is in excess of 1 CD133+ cell in a total of 10⁴ cells.

9. The test of claim 1, wherein the test is considered positive for the existence of a cancerous condition when the amount of CD133+ cells in the sample is in excess of 1 CD133+ cell in a total 10⁵ cells.

10. The test of claim 1, wherein the cells in the sample are so treated as to separate individual cells in the sample.

11. The test of claim 1, wherein the cells in the sample are so treated as to separate individual cells in the sample, such that at least 10% of the cells of the treated sample are associated with no more than one other cell.

12. The test of claim 1, wherein the cells in the sample are so treated as to separate individual cells in the sample, such that at least 25% of the cells of the treated sample are associated with no more than one other cell.

13. The test of claim 1, wherein the cells in the sample are so treated as to separate individual cells in the sample, such that at least 50% of the cells of the treated sample are associated with no more than one other cell.

14. The test of claim 9, wherein the cells in the same are treated as to separate individual cells in the sample such that none of the cells is associated with another cell.

15. The test of claim 1, wherein the presence of the CD133 marker is detected by at least one of microscopic observation, radioimmunoassay measurement, chromophore detection, ligand binding immunoassay, magnetic labelling immunoassay, enzyme-linked or other immunoassay, chromatography, FACS (fluorescence activated cell sorting) and flow cytometry.

16. The test of claim 1, wherein non-cancerous cells are removed from the tissue sample before contacting the sample with an antibody specific for CD133.

17. The test of claim 15, wherein said non-cancerous cell which are removed are fibroblasts, haematopoietic cells or endothelial cells.

18. A method for detecting the presence of cancerous tissue in a sample from the respiratory tract, comprising contacting the sample with an optionally labelled anti-CD133 antibody, and determining the level of binding of the antibody to the sample.

19. The method of claim 18, wherein the antibody is labelled such as to fluoresce, or be prepared in the form of microbeads, or may be unlabelled but detectable on a chromatographic column.

20. A kit comprising antibodies which specifically bind CD133 protein, together with instructions for the use thereof in the diagnosis of the existence of a cancerous condition.

21. A method for cultivating CD133+ respiratory tumour cells, and especially CD133+ lung carcinoma cells, comprising the use of permissive conditions.

22. A process for the selection of potential therapeutic agents, comprising the use of the cells of claim 21 to screen for therapeutic agents effective against cancers, especially those of the respiratory tract, and especially those tumours of whom the CD133+ cells form a part.

23. A method for selecting treatment for cancer of the respiratory tract according to the results obtained from a test as defined in claim 1.
